(19) United States
(12) Patent Application Publication
(10) Pub. No.: US 2026/0248257 A1
(43) Pub. Date: Aug. 27, 2026

(54) HAIR DYEING METHOD

(71) Applicant: L'OREAL, PARIS (FR)

(72) Inventors: Gabin VIC, SAINT-OUEN (FR); Melissa LIAUZUN, CHEVILLY-LARUE (FR); Valeria NUZZO, SAINT-OUEN (FR)

(21) Appl. No.: 19/148,272

(22) PCT Filed: Jan. 22, 2024
(86) PCT No.: PCT/EP2024/051432

§ 371 Date: Jul. 15, 2025

(30) Foreign Application Priority Data
Jan. 20, 2023 (FR) FR2300543

(51) Int. Cl. A45D 19/00; A45D 2/24; A61K 8/22; A61K 8/41; A61K 8/49; A61Q 5/06

(52) U.S. Cl. CPC A45D 19/0066; A45D 2/2407; A45D 2/2464; A61K 8/22; A61K 8/41; A61K 8/492; A61Q 5/065; A45D 2200/205; A61K 2800/434; A61K 2800/87

(57) ABSTRACT

The invention relates to a cosmetic hair dyeing method comprising at least the steps consisting in:
(a) applying to at least one lock of hair an aqueous dyeing composition comprising one or more oxidation dyes and one or more nitrogenous alkaline agents,
(b) exposing the hair impregnated with the dyeing composition to microwave radiation.

### TECHNICAL FIELD

The present invention relates to hair dyeing methods.

### PRIOR ART

Numerous techniques for dyeing the hair have already been provided, employing different types of dyeing compositions.

In addition, it is known to use microwave radiation in the field of surface maintenance to increase the action of detergents. In particular, Applications WO 98/03621, WO 98/03622, WO 98/03623, WO 99/25802 and WO 99/37745 describe hard surface treatments combining exposure to microwave radiation with detergents containing bleaching agents.

In the field of hair shaping, Applications DE 3148538, US 2006/0108358, US 2010/0006116 or US 2007/056860 disclose the direct use of microwave radiation on the hair.

The documents US 2006/042649 and U.S. Pat. No. 6,079,422 describe heatable curlers which can be placed in a microwave oven before use for the winding of the hair. In these documents, it is the aspect of generation of heat by microwaves which is used but there is no direct contact of these microwaves with the hair.

Application WO 2013/183021 discloses a method for the treatment of keratin fibres comprising the application of a shaping composition followed by the application of a mechanical tension and then exposure to microwave radiation.

Applications WO 2011/141882 and FR 3 062 786 describe hair treatment methods with a step of placing under mechanical tension and a step of exposure to microwave radiation.

Application WO 2013/125052 describes a process for colouring hair using synthetic direct dyes and the heating of keratin fibres.

Application WO 2019/122850 discloses a hair dyeing method using a dyeing composition and simultaneous treatment with radio wave radiation with a frequency of between 1 MHz and 300 MHz.

The shaping of the hair and the dyeing of the same hair usually require two different operations which, on the one hand, result cumulatively in lengthy periods of immobilization and, on the other hand, are inadvisable in immediate successive applications because of the risks of denaturation of the hues obtained on the hair with, moreover, a significant increase in the risk of damage to the keratin fibres.

Consequently, in the light of these documents, there still exists a need to improve hair dyeing methods, by increasing their effectiveness while preserving the hair fibre as much as possible, and also to provide a method for simultaneously dyeing and shaping the hair.

### DISCLOSURE OF THE INVENTION

### Dyeing Method

The invention is targeted at meeting this need and has as subject-matter a cosmetic hair dyeing method comprising at least the steps consisting in:
(a) applying to at least one lock of hair an aqueous dyeing composition comprising one or more oxidation dyes and one or more nitrogenous alkaline agents,
(b) exposing the hair impregnated with the dyeing composition to microwave radiation.

The method according to the invention makes it possible to obtain an improved hair dyeing treatment, thanks to the threefold combination of oxidation dyes, nitrogenous alkaline agents and microwave radiation. The use of the latter, while the dyeing composition is still present on the hair, provides targeted excitation of the molecules, making it possible to reduce the exposure time for an equivalent result.

It is also possible to reduce the concentration of oxidation dyes and/or of nitrogenous alkaline agents used to achieve a given level of colouration.

The dyeing results are very good with in particular good coverage of white hair, good persistence and low selectivity, while limiting the damage inflicted on the hair.

The use of microwave radiation makes it possible to accelerate the dyeing reaction and thus to reduce the leave-on time necessary for the composition.

The durability and homogeneity of the results obtained from the method according to the invention are very good, and the damage experienced by the hair is reduced and the risks of irritation limited.

Different results are obtained depending on the duration of exposure to microwave radiation, depending in particular on the base colour of the lock of hair.

The duration of the exposure to microwave radiation can range from 1 second to 30 minutes, better still from 1 second to 10 minutes, even better still from 1 second to 5 minutes, very preferentially from 1 second to 3 minutes.

The method according to the invention can additionally comprise a hair shaping step (d), comprising in particular the application of a mechanical tension to said one lock of hair, via in particular a device for application of mechanical tension, having a receiving surface intended to receive the lock of hair, in particular a step of winding the lock of hair on a curler.

The method according to the invention can thus contribute in parallel to a degree of shaping of the hair, and also offers the possibility of carrying out simultaneously the dyeing and shaping treatments, in a lasting, rapid and satisfactory manner. The curls are well defined and sufficiently durable, with limited damage.

In this alternative form, the shaping step (d) can be carried out before step (a) of application of the composition or between step (a) and step (b) of exposure to microwave radiation.

The method according to the invention can additionally comprise one or more pretreatment steps taking place before steps (a) and (b), and/or before or after step (d), if appropriate.

The method according to the invention can additionally comprise one or more post-treatment steps taking place after step (b), and/or after step (c), if appropriate.

These additional pretreatment or post-treatment steps can comprise the application of one or more additional compositions other than that present during step (a). The application of these additional compositions may or may not be immediately followed by a microwave treatment.

In the case where a shaping step (d) is carried out, the method according to the invention can comprise a step (e) of application of a hair-shaping composition comprising either one or more film-forming fixing polymers, also called fixing polymers, or one or more agents which break the disulfide bridges of keratin, such as thiol-containing reducing agents. Preferably, the method according to the invention does not comprise such a step (e).

The method according to the invention can additionally comprise one or more steps (c) of rinsing the hair, after one or other of steps (a), (b) and (d), preferably a single rinsing step carried out after step (b) of exposure to microwave radiation.

### Composition

The dyeing composition preferably comprises at least 5% by weight of water at ambient temperature (25° C.) and at atmospheric pressure (760 mm of mercury).

The dyeing composition can result from a mixture of several compositions, being in particular the mixture of a composition comprising one or more oxidation dyes and one or more nitrogenous alkaline agents (a1*) and of an aqueous composition (a1**) comprising in particular at least one oxidizing agent. The aqueous composition (a1**) can itself result from the mixing of two or more compositions. The mixing of the compositions is preferably carried out at the time of the use of the dyeing composition.

Preferably, the dyeing composition (a1) comprises at least one oxidizing agent.

Preferably, the composition (a1) comprises hydrogen peroxide. This hydrogen peroxide can originate from an anhydrous hydrogen peroxide precursor, such as urea peroxide, then present in a composition which hydrolyses in the presence of the composition (a1*) or directly in the composition (a1). More preferentially, the hydrogen peroxide can be in the form of an aqueous hydrogen peroxide solution present in the composition (a1**).

In an alternative form, the composition (a1) is devoid of oxidizing agents. In this alternative form, the method according to the invention advantageously comprises a step (f) of application of an oxidizing composition after step (b), said oxidizing composition then preferably containing hydrogen peroxide in an aqueous medium.

Preferably, the dyeing composition (a1) has a pH greater than 7, ranging in particular from 7.1 to 13, better still from 7.5 to 12, even better still from 8 to 11.

One or more nitrogenous alkaline agents are contained in the composition (a1).

An agent is regarded as alkaline when, introduced into water at ambient temperature and at atmospheric pressure, it results, at the concentration by weight of 1%, in a pH greater than 7.

The nitrogenous alkaline agent(s) contained in the dyeing composition (a1) are preferably chosen from ammonia and alkanolamines, in particular monoethanolamine.

In the dyeing composition (a1), the concentration of nitrogenous alkaline agents can vary from 0.01% to 20% by weight, better still from 0.1% to 15% by weight, even better still from 0.5% to 10% by weight, with respect to the total weight of the composition.

Preferably, the dyeing composition (a1) comprises one or more oxidation bases preferentially chosen from para-phenylenediamines, para-aminophenols and heterocyclic bases, such as pyrazoles.

More preferentially still, the dyeing composition (a1) comprises one or more couplers, preferably chosen from meta-diphenols, meta-aminophenols, meta-phenylenediamines or heterocyclic couplers, such as indoles.

In the dyeing composition (a1), the concentration of oxidation dyes can vary from 0.001% to 20% by weight, better still from 0.01% to 15% by weight, even better still from 0.1% to 10% by weight, with respect to the total weight of the composition.

The cosmetic compositions used in the method according to the invention can also comprise at least one compound chosen from solvents, silicone or non-silicone fatty substances, silicone or non-silicone anionic, cationic, non-ionic or amphoteric surfactants, polymers and in particular anionic, cationic, non-ionic or amphoteric treating or thickening polymers, anti-dandruff agents, or agents for combating hair loss.

### Microwave Radiation

The term “microwaves” should be understood as meaning electromagnetic radiation with a frequency ranging from 500 MHz to 300 GHz.

Preferably, the frequency of the microwave radiation used in the method of the invention ranges from 500 MHz to 300 GHz, better still from 500 MHz to 10 GHz, even better still from 900 MHz to 5 GHz, very preferentially from 2 GHz to 3 GHz.

The power of the microwaves used during step (b) can be less than or equal to 500 W, preferably ranging from 10 to 300 W, better still from 100 to 250 W, even better still from 10 to 50 W.

Preferably, the surface power density on the hair, induced by microwave radiation, can range from 0.1 W/cm² to 100 W/cm².

Exposure to microwave radiation can be carried out by virtue of a microwave emission antenna, said antenna and at least a part of the lock to be treated preferably being inserted into an enclosure leaktight to microwaves.

If appropriate, the enclosure, at least during step (b), contains the hair to be treated impregnated with the dyeing composition. The term “containing the hair” should be understood to mean containing the hair over all or part of its length and over a given width. The enclosure can cover the hair over a length, for example, greater than or equal to 5 cm. Thus, a length of at least 5 cm of hair can be treated in one go in the enclosure.

Said enclosure can be defined at least partially by a case exhibiting a housing, configured to receive at least a part of the hair to be treated and, if appropriate, a hair-shaping tool, and by a cover pivotable with respect to the case between an open position giving access to the housing and a closed position, the microwave emission antenna being positioned inside the housing.

In an alternative form, the enclosure is in the form of a helmet.

The enclosure can be immobile with respect to the hair treated during the emission of microwaves or mobile with respect to the hair, making it possible to treat a greater length of hair.

The enclosure can comprise at least one seal of an electrically conductive material, for example which is elastically deformable, making it possible to screen the microwaves used during step (b) while allowing the hair to come out of the enclosure. The seal can comprise a foam charged with electrically conductive particles, a brush formed of electrically conductive bristles, or a comb comprising metal teeth.

In the case where the enclosure is in the form of a helmet, the enclosure can have electromagnetic shielding through which the treated hair can pass. Such shielding makes it possible to treat the hair of the user while protecting the skull of the latter from the microwaves emitted. The electromagnetic shielding is formed, for example, by a metal grid or mesh.

### Assembly

According to another of its aspects, another subject-matter of the invention is an assembly for dyeing the hair comprising at least one dyeing composition, comprising one or more oxidation dyes and one or more nitrogenous alkaline agents, and a device suitable for exposing the hair to microwave radiation as defined above.

Said device can comprise a microwave emission antenna, said antenna and at least a part of the lock to be treated being in particular inserted into an enclosure leaktight to microwaves, as described above.

The microwaves can be generated inside said device suitable for exposing the hair to microwave radiation or outside by a microwave generator, for example a solid state generator, such as a magnetron.

In an alternative form, the generator is located inside the enclosure.

The emission of the microwaves can be carried out by a non-rectilinear antenna, for example of helical type.

The antenna can be formed of a wire which is sufficiently rigid, for example with a diameter ranging from 1 to 3 mm, so as not to require maintenance or a support rod for the turns. In an alternative form, the antenna comprises a flexible wire or a conductive track positioned on a support structure, in particular a helical support structure. This support structure can be made of any dielectric material, preferably made of PTFE or of glass.

The antenna, when it is helical, preferably has a fixed helical pitch, for example ranging from 4 to 5 mm. The radius of the helix is preferably constant, for example ranging from 4 to 10 mm. The diameter of the antenna is, for example, from 8 to 10 mm.

The axial extension of the antenna can vary from 50 to 80 mm, better still from 65 to 75 mm.

The antenna can be positioned in a tube, in particular closed at one end. This tube can be made of PTFE or of another dielectric material.

The antenna can also be embedded in a dielectric material, preferably a flexible dielectric material, in particular silicone.

Said device suitable for exposing the hair to microwave radiation can comprise a hair-shaping tool, in particular a tool for application of a mechanical tension, in particular a curler. The term “curler” denotes a tool which makes possible the winding of the hair around the longitudinal axis of the device according to a defined diameter, thus creating a mechanical tension, which is a function of the diameter. The diameter of said curler can be a function of the curl effect desired.

The curler can exhibit teeth at the ends of branches which laterally delimit the surface for receiving the hair, said teeth of the curler advantageously being positioned around the antenna.

The shaping tool can be configured, if appropriate, so as to place the treated hair down flat during the exposure to microwaves.

The removal of the mechanical tension on said lock of hair is preferably carried out after step (b) of exposure to microwave radiation.

The device can comprise a housing capable of receiving the shaping tool. Said housing can be coated with a conductive foam which is positioned so as to extend into a hair outlet slot. This makes it possible to obtain good leaktightness of the closure, by virtue of the deformability of the foam at the slot.

If appropriate, the shaping tool and the antenna form a unitary assembly which is introduced into the enclosure.

The shaping tool can be made of PTFE or of other dielectric materials, especially thermoplastic dielectric materials.

The device used to implement the method according to the invention can comprise a sound and/or light warning system, for example to warn the user of an escape of microwaves outside the enclosure and/or of an excessive temperature inside the enclosure.

The device advantageously comprises a safety system which prevents the emission of microwaves while the enclosure is not closed and/or in the event of abnormal operation, for example of escape of microwaves and/or of excessive temperature.

The device can be configured to control the duration of emission of the microwaves, in order not to reach a duration of treatment liable to damage the hair.

The method according to the invention can comprise, before step (b), a step of detection of the closure of the enclosure. For example, a switch is actuated when the enclosure is closed.

The start of the microwaves can be dependent on the detection of the closure of the enclosure. After start of the microwaves, the continuation of the emission of the microwaves can be dependent on the absence of detection of escape of microwaves, in particular by means of a sensor sensitive to microwaves placed outside the enclosure.

The method according to the invention can additionally comprise a step of detection of the positioning of the hair intended to be treated, before step (b). This detection step can be carried out, for example, by an optical sensor and/or a mechanical feeler. If appropriate, detection can be carried out indirectly, by detecting the positioning of the shaping tool.

The method according to the invention can comprise, for example during step (b), a step of measurement of the temperature to which the treated hair is subjected. This step of measurement of the temperature can be carried out by a thermometer without contact with the hair.

The device can comprise, within one and the same handpiece, the microwave generator and also the shaping tool. The term “handpiece” denotes a part manipulated by the user with one hand during the treatment of the hair.

The device can comprise a sensor sensitive to a characteristic of the individual hair, for example the colour, the mechanical strength, the surface condition or the humidity, and the device can control at least one parameter of the treatment as a function of the characteristic thus detected, for example the energy of the microwaves, the temperature of the solvent, the duration of the treatment and/or the mechanical stress exerted, if appropriate.

The characteristics set out above for the method apply to the assembly, and vice versa.

### BRIEF DESCRIPTION OF THE DRAWINGS

A better understanding of the invention will be obtained on reading the description which follows and on examining the figures which accompany it. These are presented only by way of indication and without in any way limiting the invention.

FIG. 1 illustrates various steps of an example of the hair dyeing method according to the invention,

FIG. 2 diagrammatically and partially represents an example of a device suitable for implementing the method according to the invention,

FIG. 3 represents the handpiece, in the open position, of an example of a device suitable for implementing the method according to the invention,

FIG. 4 represents the handpiece of FIG. 3, in the closed position,

FIG. 5 represents a shaping tool, once extracted from the handpiece of an alternative form of a device suitable for implementing the method according to the invention, and

FIG. 6 is a cross-sectional and side view of a device suitable for implementing the method according to the invention and comprising the shaping tool of FIG. 5, when the enclosure is closed.

### DETAILED DESCRIPTION

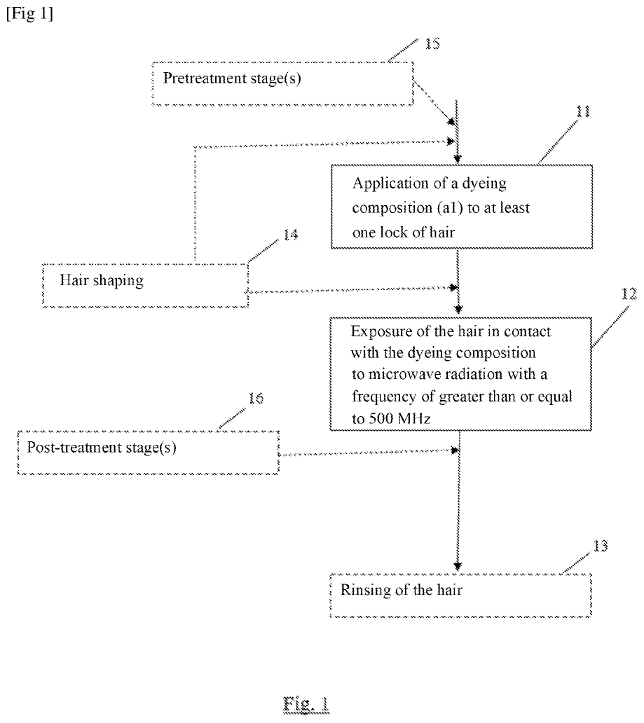

Various steps or stages of an example of the hair dyeing method according to the invention are represented in FIG. 1 and will be described in detail in that which follows.

In this example, during a step 11, an aqueous dyeing composition (a1) comprising one or more oxidation dyes and one or more nitrogenous alkaline agents is applied to at least one lock of hair.

In a step 12, the hair in contact with the dyeing composition is exposed to microwave radiation with a frequency greater than or equal to 500 MHz, preferably ranging from 900 MHz to 5 GHz. The duration of the exposure to microwave radiation preferably ranges from 1 second to 3 minutes. The surface power density on the hair, induced by microwave radiation, can range from 0.1 W/cm² to 100 W/cm².

The method according to the invention additionally comprises, in the example described, one or more hair rinsing steps 13, preferably a single hair rinsing step 13, carried out after step 12 of exposure to microwave radiation.

The method according to the invention can additionally comprise a hair shaping step 14, comprising in particular the application of a mechanical tension to said at least one lock of hair, via a device for application of mechanical tension, having a receiving surface intended to receive the lock of hair, in particular a step of winding the lock of hair on a curler. This shaping step 14 is carried out before step 11 of application of the dyeing composition or between steps 11 and 12.

The method according to the invention can additionally comprise one or more pretreatment steps 15 which is/are carried out before steps 11 and 12, for example comprising the application of one or more additional compositions other than that used during step 11.

One or more post-treatment steps 16 can be carried out after step 12 and/or after step 13, for example comprising the application of one or more additional compositions other than that used during step 11.

Preferably and as in the example described, the dyeing composition (a1) comprises hydrogen peroxide. The dyeing composition can be a mixture of several compositions, being in the example considered the mixture of a composition comprising one or more oxidation dyes and one or more nitrogenous alkaline agents (a1*) and of an aqueous composition (a1**) comprising said hydrogen peroxide, the mixing of the compositions being carried out at the time of the use of the dyeing composition. The oxidation dyes and the nitrogenous alkaline agents included in the dyeing composition (a1) and their concentration are as described above.

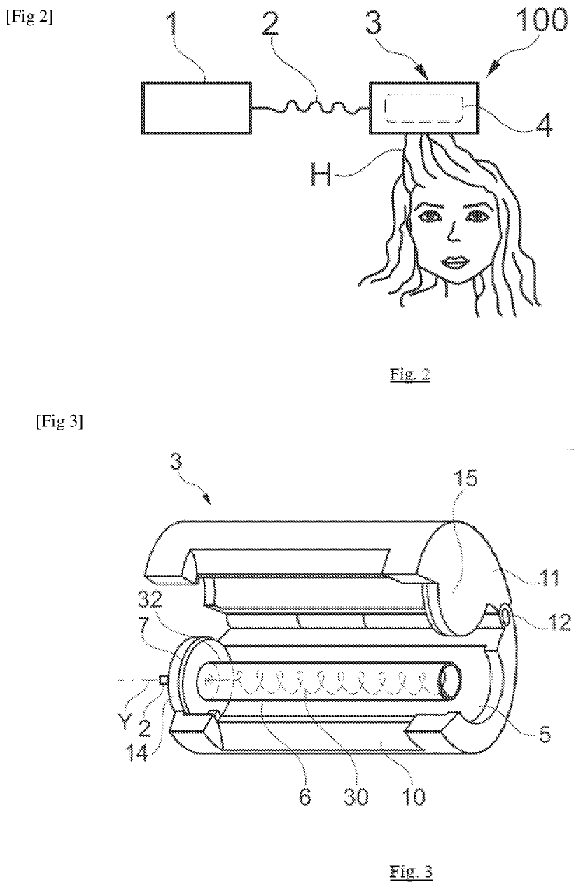

A device 100 suitable for the implementation of the method according to the invention, comprising a handpiece 3 connected to a base unit 1, comprising a microwave generator, by a flexible waveguide 2, has been represented in FIG. 2. The handpiece 3 is expected to be manipulated by the user in order to be placed close to the head of the person in order to treat at least one lock of hair H, as illustrated. The handpiece 3 can be made in various ways and defines a treatment enclosure 4 which is leaktight to microwaves.

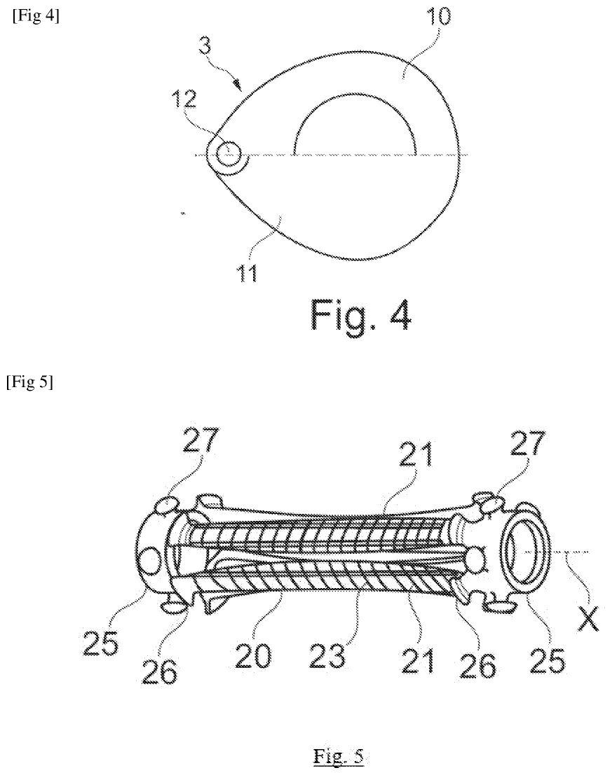

In the example represented in FIG. 3, the handpiece 3 comprises a case 10, defining a housing 6, on which a cover 11 is articulated by means of an axis 12. The handpiece is represented closed in FIG. 4. The flexible waveguide 2 has been represented only very partially in FIG. 3 et seq.

The case 10 carries an antenna 30 which, in the example illustrated, is helical in shape, with a longitudinal axis 7.

In the example considered, the antenna 30 is composed of a helical metal wire, positioned in a tube 32 of a dielectric material, for example silicone or PTFE.

The antenna 30 is supported at one axial end of the case 10 by an end plate 14 of the case 10, of semi-circular outline.

The cover 11 exhibits a similar end plate 15 on the opposite side, in order to close the opening 5 of the housing 6, by which opening a shaping tool can be introduced into the handpiece 3.

The inner surface of the housing 6 is defined by an electrically conductive coating 7 which makes it possible to reflect the microwaves and makes the enclosure 4 leaktight to microwaves. This coating 7 can be formed of a conductive foam, for example of semi-cylindrical shape, on the cover and the case, in the form of a disc for the end plate 15 and of a ring for the end plate 14.

The case 10 and the cover 11 define an outlet slot 19 for the hair, located at the bottom of a recess 18, on the side opposite the axis 12 of the hinge.

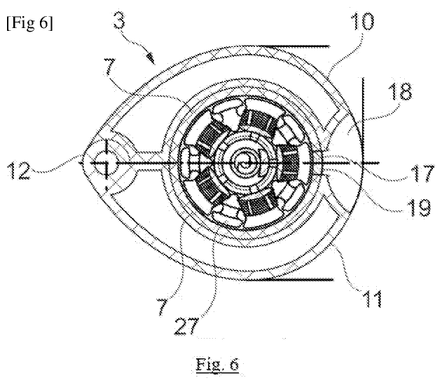

The coating 7 preferably protrudes into the slot 19, as can be seen in FIG. 6, so as to produce good leaktightness of the closure despite the passage of the hair. The edge of the coating can thus slightly exceed at 17 the edge of the body of the cover and that of the case to close the slot 19.

The handpiece 3 comprises a member (not represented) for locking the cover in the closed position, and also an opening detector.

A temperature detector can be present within the enclosure 4, for example on an inner surface of the latter, in order to measure the temperature of the lock of hair treated, and a control system, for example having a microprocessor, can make it possible to interrupt or modulate the emission of microwaves in the case of a detected temperature greater than a predefined threshold.

In a particular embodiment, the case 10 defines a housing 6 for receiving a shaping tool consisting, in the example considered, of a curler 20, represented in FIG. 5. The presence of the tube 32, represented in FIG. 3, wherein the wire of the antenna 30 is positioned, facilitates the insertion of the antenna inside the curler 20.

The latter comprises several branches 21 which define a surface for receiving the hair H, of width L, measured along the longitudinal axis X of the curler 20.

In this example, the axis X is parallel to the axis of rotation of the cover 11 and coincident with the longitudinal axis 7 of the antenna 30.

In the example considered, the curler 20 comprises four branches 21, which are slightly curved towards the axis X and pass through a minimum of distance to the axis X at approximately mid-length.

The branches 21 can exhibit ribs 23 oriented perpendicularly to the axis X in order to help keep the lock spread out over the branches 21, preferably occupying the entire width L in the most uniform manner possible.

The branches 21 are connected at their ends to annular parts 25.

The curler 20 exhibits teeth 26 at the ends of the branches 21, which laterally delimit the surface for receiving the hair.

The curler 20 can be made of any dielectric material, preferably PTFE.

The curler 20 preferably exhibits a symmetrical shape of revolution with respect to a median plane of symmetry perpendicular to the axis X.

The curler 20 comprises studs 27 having an enlarged head, which are connected at their base to the corresponding annular parts 25 and which are supported on the coating 7, as illustrated in FIG. 6, in order to keep the curler centred in the housing 6.

The studs 27 are, for example, located between the teeth 26, as illustrated.

The width W of the outlet slot 19 for the hair can be slightly greater than L, the length of the housing 6 being substantially equal to the length of the curler 20, so that the curler is immobilized both axially and radially in the housing 6, apart from a slight play.

To use the handpiece 3, a lock of hair impregnated with dyeing composition is wound onto the curler 20, then the curler is placed inside the case 10 by moving it laterally relative to the latter, it being introduced through the opening 5. Subsequently, the cover 11 is closed again and the hair comes out through the slot 19. Once exposure to microwaves has been carried out, the lock of hair is extracted by carrying out the operations in the reverse order.

### Example

An example of results provided by the method according to the invention are summarized in Table 1 below.

In this example, dyeing tests were carried out on hair of Caucasian type with a colour depth level equal to 6 (DL6) and on hair comprising 90% natural white hairs (NW90).

The compositions used in this example are aqueous dyeing compositions (a1) according to the invention, resulting from a mixture of a composition comprising one or more oxidation dyes and one or more nitrogenous alkaline agents (a1*) and of an aqueous composition (a1**) comprising at least one oxidizing agent, the mixing of the compositions being carried out at the time of the use of the dyeing composition (proportion 1 for 1 in preparing 10 g of mixture per gram of hair). More particularly, the compositions used are commercial oxidation dyeing products of the Majirel brand, combined with a 20 volume oxidant of the L'Oreal Professional brand. The references 6.66, 7.3 and 5 were used, corresponding respectively to an intense red, a golden blond and a light chestnut. In addition to oxidation bases and couplers, these Majirel dyeing products include nitrogenous alkaline agents such as ethanolamine and ammonia (ammonium hydroxide). For example, in reference 6.66, the concentration of ethanolamine is equal to 0.63%.

The dyeing composition (a1) was applied to both faces of a lock of hair with a brush, the lock thus impregnated being subsequently exposed for 3 minutes to microwave radiation with a power of 30 W.

The colorimetric parameters observed are the ΔE, corresponding to the measurement of the variation in the visual perception of two given colours, before and after treatment by the method of the invention, comparable to the increase in colouration, and the variation in chromaticity, before and after treatment by the method of the invention.

The results obtained by virtue of the employment of the method according to the invention were compared with results obtained by employing a dyeing method of the prior art, not using microwave radiation, the hair impregnated with dyeing composition being left at ambient temperature (AT) for 30 minutes.

TABLE 1

| Hair and method used | ΔE | CHROMATICITY |
|---|---|---|
| Prior art: NW90 6.66 AT 30 | 42.98 | 15.6 |
| min | | |
| Invention: NW90 6.66μ 30 | 43.11 | 14.2 |
| W 3 min | | |
| Prior art: DL6 6.66 AT 30 | 17.48 | 10.4 |
| min | | |
| Invention: DL6 6.66μ 30 W | 17.19 | 11.4 |
| 3 min | | |
| Prior art: NW90 5 AT 30 min | 43.68 | 2.4 |
| Invention: NW90 5μ 30 W 3 | 42.05 | 4.2 |
| min | | |
| Prior art: DL6 5 AT 30 min | 21.44 | 1.6 |
| Invention: DL6 5μ 30 W 3 | 20.29 | 2.5 |
| min | | |
| Prior art: NW90 7.3 AT 30 | 36.46 | 10.4 |
| min | | |
| Invention: NW90 7.3μ 30 W | 34.64 | 11.25 |
| 3 min | | |
| Prior art: DL6 7.3 AT 30 min | 12.03 | 9.9 |
| Invention: DL6 7.3μ 30 W 3 | 10.13 | 11.1 |
| min | | |

Compared with the method of the prior art, the colorimetric parameters observed remain comparable, the treatment time necessary in the method according to the invention being, on the other hand, reduced by a factor of 10.

Numerous modifications can be made to the methods and devices which have just been described, without departing from the scope of the present invention.

The invention is not limited to a particular type of shaping tool or to a particular type of microwave emission antenna.

1-17. (canceled)

18. A method for dyeing hair, comprising:
(a) treating the hair by applying to the hair an aqueous dyeing composition (a1) comprising: one or more oxidation dyes and one or more nitrogenous alkaline agents; and
(b) exposing the treated hair to microwave radiation.

19. The method of claim 18, wherein the frequency of the microwave radiation ranges from 500 MHz to 300 GHz.

20. The method of claim 18, comprising:
(d) shaping the hair, comprising:
(i) applying a mechanical tension to the hair via a device, wherein the device comprises a receiving surface configured to receive the hair, and
(ii) winding the hair on a curler,
wherein step (d) is carried out before step (a), or between step (a) and step (b).

21. The method of claim 18, further comprising a step (c) of rinsing the hair, wherein step (c) is carried out after any one or more of steps (a), (b), or (d).

22. The method of claim 18, wherein the aqueous dyeing composition (a1) is a mixture of a composition (a1*) with a composition (a1**),
wherein composition (a1*) comprises one or more oxidation dyes and one or more nitrogenous alkaline agents,
wherein composition (a1**) comprises at least one oxidizing agent,
wherein composition (a1*) and composition (a1**) are mixed at the time of using the aqueous dyeing composition (a1).

23. The method of claim 18, wherein the aqueous dyeing composition (a1) further comprises at least one oxidizing agent.

24. The method of claim 18, wherein the aqueous dyeing composition (a1) further comprises hydrogen peroxide.

25. The method of claim 18, wherein the aqueous dyeing composition has a pH greater than 7.

26. The method of claim 18, wherein the one or more nitrogenous alkaline agents of aqueous dyeing composition (a1) is chosen from ammonia, alkanolamines, or mixtures thereof.

27. The method of claim 18, wherein the one or more nitrogenous alkaline agents of aqueous dyeing composition (a1) is monoethanolamine.

28. The method of claim 18, wherein the total amount the one or more nitrogenous alkaline agents in aqueous dyeing compositions (a1) ranges from 0.01% to 20% by weight, relative to the total weight of the composition.

29. The method of claim 18, wherein the total amount nitrogenous alkaline agents in aqueous dyeing composition (a1) ranges from 0.5% to 10% by weight, relative to the total weight of the composition.

30. The method of claim 18, wherein the aqueous dyeing composition (a1) further comprises one or more couplers chosen from meta-diphenols, meta-aminophenols, meta-phenylenediamines, heterocyclic couplers, or mixtures thereof.

31. The method of claim 30, wherein the heterocyclic couplers are chosen from indoles.

32. The method of claim 18, wherein the total amount of one or more oxidation dyes in aqueous dyeing composition (a1) ranges from 0.001% to 20% by weight, relative to the total weight of the composition.

33. The method of claim 18, wherein exposure to microwave radiation is carried out via a microwave emission antenna, wherein the microwave emission antenna and at least a part of the hair are inserted into an enclosure which is leaktight to microwaves.

34. The method of claim 18, wherein the surface power density on the hair, induced by microwave radiation, ranges from 0.1 W/cm² to 100 W/cm² and wherein the duration of the exposure to microwave radiation ranges from 1 second to 30 minutes.

35. An assembly for dyeing hair comprising:
at least one dyeing composition comprising one or more oxidation dyes and one or more nitrogenous alkaline agents; and
a device configured to expose the hair to microwave radiation with a frequency ranging from 500 MHz to 300 GHz.

36. The assembly of claim 35, wherein the device comprises a microwave emission antenna, wherein the microwave emission antenna and at least a part of the hair are inserted into an enclosure which is leaktight to microwaves.

37. The assembly of claim 35, wherein the device comprises a hair shaping tool.
